(19) United States
(12) Patent Application Publication
(10) Pub. No.: US 2026/0345228 A1
(43) Pub. Date: Nov. 5, 2026

(54) ASPIRATING DIATHERMY PROBE

(71) Applicant: Alcon Inc., Fribourg (CH)

(72) Inventors: Reto Grüebler, Greifensee (CH); Christoph Siegenthaler, Eschenz (CH)

(21) Appl. No.: 19/637,195

(22) Filed: Apr. 2, 2026

Related U.S. Application Data
(60) Provisional application No. 63/798,073, filed on May 1, 2025.

(51) Int. Cl. A61B 18/14; A61F 9/007; A61M 1/00

(52) U.S. Cl. CPC A61B 18/1477; A61F 9/00709; A61M 1/87

(57) ABSTRACT

In certain embodiments, a surgical device comprises a flexible aspiration tube, a diathermy electrical cable, a handpiece, and a tip attached to the handpiece. The handpiece is configured to support a distal portion of the flexible aspiration tube and a distal portion of the diathermy electrical cable. The tip comprises an aspiration tube coupled to the distal portion of the flexible aspiration tube and the distal portion of the diathermy electrical cable. The surgical device further includes an inner needle coupled to the distal portion of the diathermy electrical cable. The aspiration tube is a first diathermy electrode, and the inner needle is a second diathermy electrode.

### INTRODUCTION

The present disclosure relates to surgical systems. More particularly, the present disclosure relates to surgical systems and devices that combine diathermy and aspiration in a single surgical tool.

During an ophthalmological surgical procedure, the retina may begin bleeding into the vitreous humor of the eye. The blood may obstruct the view of the fundus (which includes the retina, macula, optic disc, fovea, and blood vessels), and cause the surgeon to pause the surgical procedure. Before continuing, the surgeon removes the blood from the vitreous humor, determines the source of the bleeding, and then stops the bleeding from the retina. For example, the surgeon may insert an aspiration probe into the eye to aspirate the blood from the vitreous humor. After a clear view of the fundus has been restored, the surgeon may determine the source of the bleeding. The surgeon then removes the aspiration probe from the eye and inserts a diathermy probe to apply an electrical stimulus to the source of the bleeding. The diathermy probe stops the bleeding by coagulating the tissue at the source of the bleeding.

### SUMMARY

In certain embodiments, a surgical device comprises a flexible aspiration tube, a diathermy electrical cable, a handpiece, and a tip attached to the handpiece. The handpiece is configured to support a distal portion of the flexible aspiration tube and a distal portion of the diathermy electrical cable. The tip comprises an aspiration tube coupled to the distal portion of the flexible aspiration tube and the distal portion of the diathermy electrical cable, and an inner needle coupled to the distal portion of the diathermy electrical cable. The aspiration tube is a first diathermy electrode, and the inner needle is a second diathermy electrode.

### BRIEF DESCRIPTION OF THE DRAWINGS

FIG. 1A illustrates an example surgical system, in accordance with certain embodiments of the present disclosure.

FIG. 1B illustrates an example surgical console, in accordance with certain embodiments of the present disclosure.

FIG. 1C illustrates a portion of the example surgical console, in accordance with certain embodiments of the present disclosure.

FIG. 2A depicts a first example aspirating diathermy probe, in accordance with certain embodiments of the present disclosure.

FIGS. 2B, 2C, 2D, and 2E depict views of the tip of the first example aspirating diathermy probe, in accordance with certain embodiments of the present disclosure.

FIG. 3A depicts a left side cross-sectional view of the distal end of a second example aspirating diathermy probe, in accordance with certain embodiments of the present disclosure.

FIG. 3B depicts a left side cross-sectional view of the distal end of a variation of the second example aspirating diathermy probe, in accordance with certain embodiments of the present disclosure.

FIG. 4A depicts a left side cross-sectional view of the distal end of a third example aspirating diathermy probe, in accordance with certain embodiments of the present disclosure.

FIG. 4B depicts a left side cross-sectional view of the distal end of a variation of the third example aspirating diathermy probe, in accordance with certain embodiments of the present disclosure.

FIGS. 5A and 5B depict a left side view and a front view (respectively) of the distal end of a fourth example aspirating diathermy probe, in accordance with certain embodiments of the present disclosure.

FIGS. 6A and 6B depict a left side view and a front view (respectively) of the distal end of a fifth example aspirating diathermy probe, in accordance with certain embodiments of the present disclosure.

FIGS. 7A, 7B, 7C, and 7D depict views of the tip of a sixth example aspirating diathermy probe, in accordance with certain embodiments of the present disclosure.

FIGS. 8A and 8B depict a left side view (partial cutaway) and a front view (respectively) of the tip of a seventh example aspirating diathermy probe, in accordance with certain embodiments of the present disclosure.

FIGS. 9A and 9B depict a left side view (partial cutaway) and a front view (respectively) of the tip of an eighth example aspirating diathermy probe, in accordance with certain embodiments of the present disclosure.

FIGS. 10A and 10B depict a left side view and a front view (respectively) of the tip of a ninth example aspirating diathermy probe, in accordance with certain embodiments of the present disclosure.

FIGS. 11A and 11B depict a left side view and a front view (respectively) of the tip of a tenth example aspirating diathermy probe, in accordance with certain embodiments of the present disclosure.

### DETAILED DESCRIPTION

Embodiments of the present disclosure will now be described with reference to the figures, in which like reference numerals refer to like parts throughout.

During an ophthalmic surgical procedure, the retina may begin bleeding into the vitreous humor of the eye, as described above. In order to stop the bleeding, the surgeon may aspirate the blood from the vitreous humor using an aspiration probe (or handpiece), determine the source of the bleeding, exchange the aspiration probe for a diathermy probe, and then coagulate the source of the bleeding using the diathermy probe. The surgeon may also use a combined irrigation/aspiration (I/A) probe in order to maintain the volume of the anterior chamber and a stable intraocular pressure (IOP) using an irrigation fluid.

The surgeon may exchange the aspiration probe and the diathermy probe several times until the bleeding is under control. For example, the bleeding may not stop completely after the initial coagulation, and another sequence of aspiration and coagulation may be needed. In another example, the bleeding may stop after the initial coagulation and then begin again at the same location on the retina after the surgeon resumes the surgical procedure. In a further example, the bleeding may stop after the initial coagulation and then begin at a different location on the retina after the surgeon resumes the surgical procedure.

The sequence of aspiration, determination, and coagulation includes inserting the aspiration probe into the eye, aspirating the blood, removing the aspiration probe from the eye, determining the source of the bleeding, inserting the diathermy probe into the eye, coagulating the source of the bleeding, and removing the diathermy probe from the eye. In other words, performing a full sequence of aspiration, determination, and coagulation may take too long and not allow for controlling the bleeding in a timely manner. For example, during the time between aspirating the blood and coagulating the source of the bleeding, the vitreous humor may fill with enough blood to obstruct the view of the fundus and prevent visualization of the retina and the source of the bleeding.

Embodiments of the present disclosure combine the functions of aspiration and coagulation into a single aspirating diathermy device (also known as a probe, a handpiece, etc.) which advantageously eliminates the need for the surgeon to change back and forth between separate aspiration and diathermy probes. Importantly, the bleeding may be stopped by coagulation even before the view of the fundus is fully restored by aspiration. In other words, the aspirating diathermy probe advantageously provides simultaneous or parallel aspiration and coagulation.

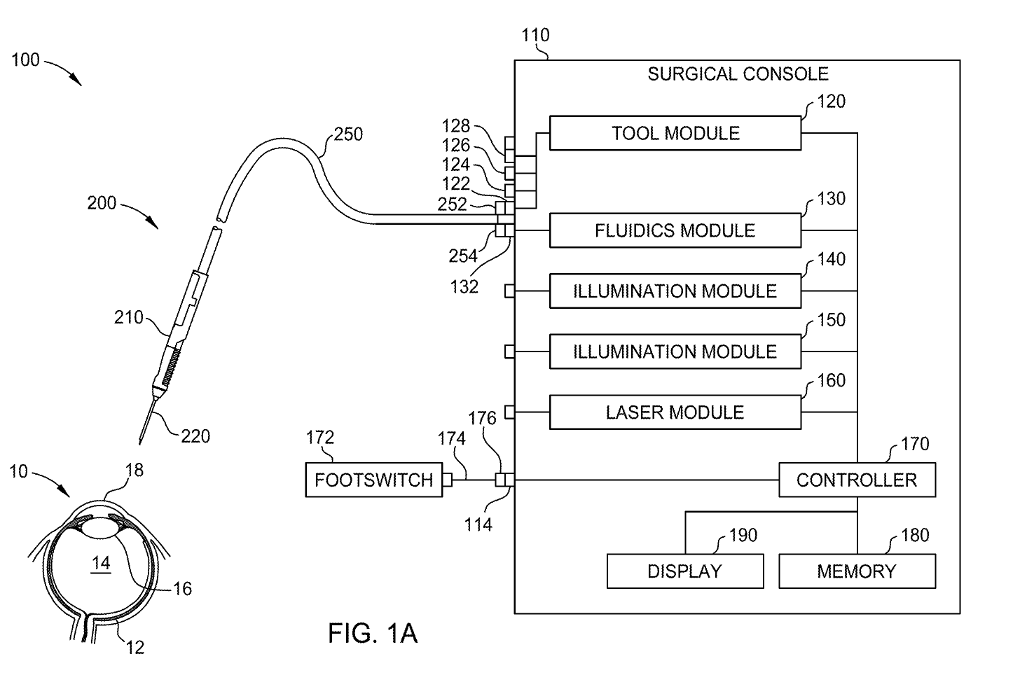

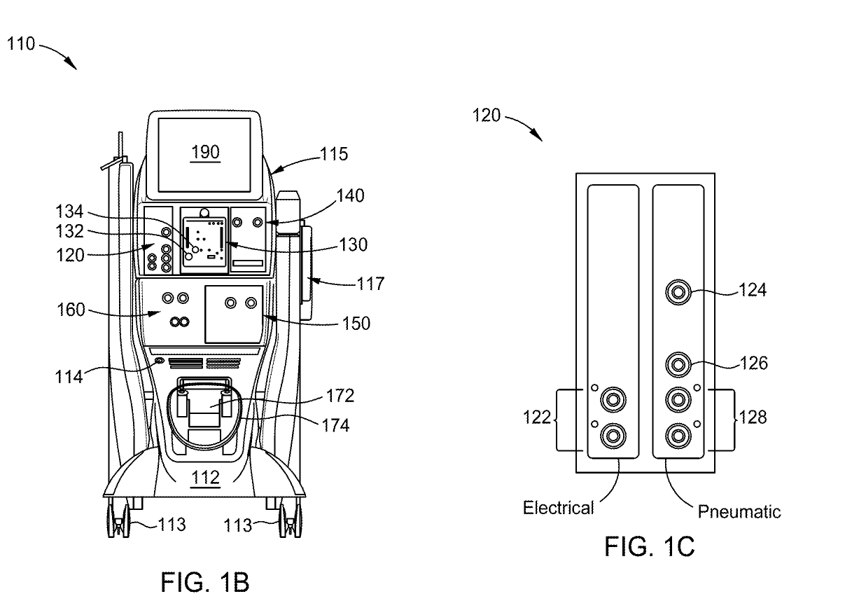

FIG. 1A illustrates an example surgical system 100 for performing an ophthalmic surgical procedure on a patient’s eye 10, in accordance with certain embodiments of the present disclosure. FIG. 1B illustrates an example surgical console 110, while FIG. 1C illustrates a portion of the example surgical console 110, in accordance with certain embodiments of the present disclosure.

Generally, the surgical system 100 may be used in a variety of ophthalmic surgical procedures that are performed on the patient’s eye 10, such as glaucoma surgery, vitreoretinal surgery, cataract surgery, etc. Additionally, the surgical system 100 may be configured to support a particular ophthalmic surgical procedure on the retina 12, the vitreous humor 14, the lens 16, the cornea 18, etc.

In certain embodiments, the surgical system 100 includes, among other things, a surgical console 110, a footswitch 172, and a surgical tool, such as an aspirating diathermy probe 200.

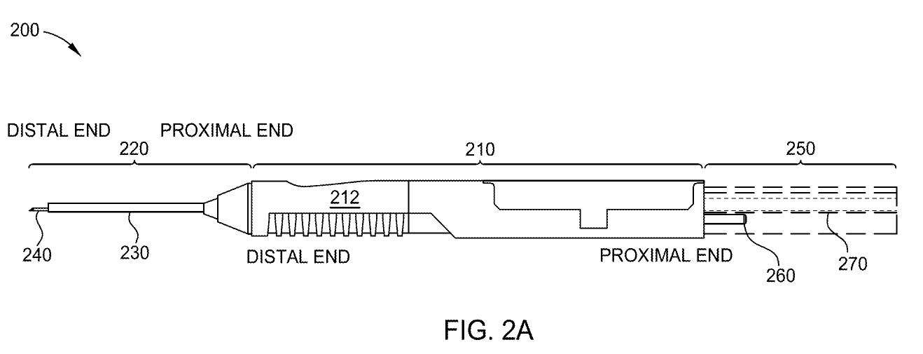

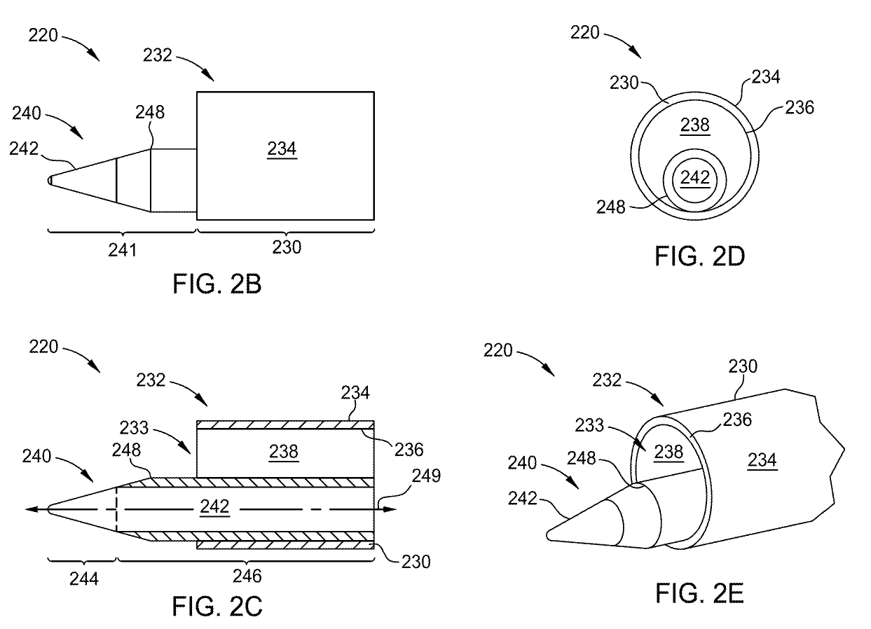

The aspirating diathermy probe 200 includes a handpiece 210, a tip 220, and a cable 250 with an electrical connector 252 and a fluidic connector 254. Embodiments of the aspirating diathermy probe 200 are discussed below with respect to FIGS. 2A-11B. The surgical system 100 may also include other surgical tools, such as a diathermy vitrectomy probe, a diathermy forceps, a diathermy scissors, etc.

The surgical console 110 may include, among other things, a tool module 120, a fluidics module 130, an illumination module 140, another illumination module 150, a laser module 160, a controller 170, a memory 180, and a touchscreen display 190. An electrical cable 174 is coupled to the footswitch 172 and includes an electrical connector 176 that is configured to connect to the footswitch electrical connector 114. As depicted in FIG. 1B, the surgical console 110 may also include a base 112 with wheels 113, a housing 115, and an articulating instrument tray 117. The surgical console 110 may also include additional tools and subsystems to facilitate the performance of the ophthalmic surgical procedure. One example of a surgical console configured for performing anterior and posterior segment ophthalmic surgery is the Constellation® Vision System available from Alcon Laboratories, Inc., Fort Worth, Texas.

The tool module 120 includes the electrical and pneumatic control systems and power sources for the surgical tools (e.g., probes, handpieces, etc.) that may be used by the surgeon during the ophthalmic surgical procedure. The front panel of the tool module 120 includes electrical and pneumatic connectors that couple the electrical cables and pneumatic tubes of the surgical tools to the respective electrical and pneumatic control systems and power sources. In certain embodiments, the tool module 120 supports aspirating diathermy probes, pneumatic forceps, pneumatic scissors, pneumatic vitrectomy probes, etc. The tool module 120 may include diathermy electrical connectors 122, a forceps pneumatic connector 124, a scissors pneumatic connector 126, and vitrectomy probe pneumatic connectors 128.

The tool module 120 includes a diathermy electrical power supply that provides a variable output voltage that is selectable by the surgeon. In certain embodiments, the surgeon may select an output power control setting between 0 and 100 (%), which may provide, for example, between 0 W (Watts) and about 10 W of electrical power into a, for example, 75 Ohm load. (Other power and other resistances are also contemplated.) In the example provided above, the relationship between the output power control setting and the output power may be generally linear, so an output power control setting of 50 % provides about 5 W of output power. Diathermy electrical connectors 122 are coupled to the diathermy electrical power supply. The diathermy electrical connectors 122 include two electrical terminals that support bipolar (i.e., 2 electrode) diathermy probes (such as aspirating diathermy probe 200), and each electrode is coupled to an electrical terminal when the diathermy probe is connected to the diathermy electrical connectors 122. The controller 170 controls the output voltage based on the operation of the footswitch 172 by the surgeon.

The diathermy electrical connectors 122 are coupled to the electrical connector 252 when the aspirating diathermy probe 200 is connected to the surgical console 110.

The tool module 120 also includes one or more pneumatic power sources, such as a constant air pressure source, a pulsed air pressure source, etc., that provide air pressure to power one or more pneumatic tools, such as a pneumatic forceps, a pneumatic scissors, a vitrectomy probe, etc. The pneumatic connectors 124, 126, 128 are coupled to the pneumatic power source(s), and each pneumatic connector provides an air supply to a different pneumatic tool. In some embodiments, an external air pressure source may be coupled to the surgical console 110 to provide compressed air to the tool module 120.

The fluidics module 130 includes the fluidic control systems and sources (such as irrigating fluid sources, vacuum sources such as aspiration pumps, etc.) for the surgical tools that may be used by the surgeon during the ophthalmic surgical procedure. The front panel of the fluidics module 130 includes fluidic connectors that couple the fluidic tubes of the surgical tools to the respective fluidic control systems and sources. In certain embodiments, the fluidics module 130 supports aspirating diathermy probes 200, aspiration probes, irrigation probes, I/A probes, etc. As depicted in FIG. 1B, the fluidics module 130 includes at least one aspiration port 132 and an irrigation port 134.

The aspiration port 132 is coupled to the fluidic connector 254 when the aspirating diathermy probe 200 is connected to the surgical console 110.

In some embodiments, the fluidics module 130 may be configured to receive a consumable cassette that is configured to regulate the irrigating fluid, aspirate debris, monitor irrigation and aspiration pressure, and deposit the debris in a sealed drainage bag for disposal. The consumable cassette includes the fluidic connectors, such as the aspiration port 132, the irrigation port 134, etc.

The illumination module 140 is configured to generate light that is formed into one or more illumination light beams. In certain embodiments, the illumination module 140 includes a xenon arc lamp that is coupled to two independently controlled optical ports. An illumination probe may be coupled to each optical port using a fiber optical cable. The controller 170 may independently control the light emitted by each optical port.

In some embodiments, the illumination module 140 includes three continuous wave (CW) lasers that generate laser light beams in the red, green, and blue spectrums in response to control signals provided by the controller 170. The red, green, and blue laser light beams are optically combined to form a white (or pseudo-white) illumination light beam (or some other user- or system-selected color) in the visible spectrum (e.g., about 380 nm (nanometers) to 750 nm). Other visible spectrum (color) combinations may also be used. In some other embodiments, the illumination module 140 includes three light emitting diodes (LEDs) that generate light beams in the red, green, and blue spectrums in response to control signals provided by the controller 170. The red, green, and blue light beams are optically combined to form a white (or pseudo-white) illumination light beam (or some other user- or system-selected color) in the visible spectrum. In some embodiments, the illumination module 140 includes a single CW laser or LED that generates a monochromatic light beam, such as a light beam in the red spectrum, the green spectrum, the blue spectrum, etc.

The illumination module 150 is similar to the illumination module 140 and is configured to generate light that is formed into one or more illumination light beams. In certain embodiments, the illumination module 140 includes a xenon arc lamp that is coupled to two independently controlled optical ports. An illumination probe may be coupled to each optical port using a fiber optical cable. In some embodiments, the illumination module 150 may include lasers, LEDs, etc., as described above with respect to the illumination module 140.

The laser module 160 may include a diode laser that is configured to generate a laser treatment beam for various ophthalmic surgical procedures, such as retinal and macular photocoagulation, iridotomy and anterior segment procedures, trans-scleral cyclophotocoagulation, laser trabeculoplasty, trans-scleral retinal photocoagulation, and other laser treatments.

The controller 170 is coupled to the footswitch electrical connector 114, the memory 180, and the touchscreen display 190. The controller 170 may be a microcontroller, a microprocessor, a programmable logic controller (PLC), a field programmable gate array (FPGA), an application specific integrated circuit (ASIC), etc. Generally, the controller 170 is configured to control the configuration and operation of the surgical console 110 through user inputs received from the footswitch 172 and the touchscreen display 190.

The footswitch 172 may be used to control both the electrical (diathermy) and fluidic (aspiration) functions of the aspirating diathermy probe 200. The footswitch 172 includes a number of switches that are configured by the controller 170 to perform various functions during the surgical procedure, such as a foot pedal (or treadle), a left horizontal/vertical switch, a right horizontal/vertical switch, a left heel switch, and a right heel switch. Generally, the foot pedal may be used to provide control of a parameter (such as aspiration vacuum) with the foot pedal’s angle of depression proportional to the parameter output. In some modes of operation of the surgical console 110, the foot pedal may be used to provide on/off control of a function (such as diathermy), with any depression resulting in a fixed output of its parameter.

The display 190 may include a touchscreen that presents different user interface screens to the surgeon. The controller 170 generates each user interface screen, which may include multiple windows, panels, tabs, menus, etc., each of which may include buttons, icons, diagrams, schematics, etc. The touchscreen display 190 accepts user input, which is provided to the controller 170 to configure and control the various functions of the surgical console 110.

FIG. 2A depicts the aspirating diathermy probe 200, in accordance with certain embodiments of the present disclosure.

In certain embodiments, the aspirating diathermy probe 200 includes, among other things, a handpiece 210, a tip 220, and a cable 250 that includes a diathermy electrical cable 260 and a flexible aspiration tube 270.

The handpiece 210 has an outer gripping surface 212 for the surgeon and includes a passageway (not visible) configured to support the distal portions of the diathermy electrical cable 260 and the flexible aspiration tube 270. The passageway extends from the proximal end of the handpiece 210 to the tip 220 attached to the distal end of the handpiece 210.

The tip 220 includes an aspiration tube 230 and an inner needle 240 disposed within the aspiration tube 230.

The aspiration tube 230 is made from electrically conductive material. The aspiration tube 230 is fluidically coupled to the flexible aspiration tube 270 and electrically coupled to one of the insulated conductors of the diathermy electrical cable 260. The aspiration tube 230 includes a passageway through which aspirated material may be removed and functions as one of the electrodes for bipolar diathermy.

The inner needle 240 is electrically coupled to the other insulated electrical conductor of the diathermy electrical cable 260. The inner needle 240 has an insulated electrode that is made from an electrically conductive material, such as tungsten, copper, silver, stainless steel, etc., and functions as the other electrode for bipolar diathermy.

The cable 250 includes a diathermy electrical cable 260 connected to the electrical connector 252 (FIG. 1A), and a flexible aspiration tube 270 connected to the fluidic connector 254 (FIG. 1A). The diathermy electrical cable 260 includes two insulated electrical conductors, such as insulated copper wires, insulated tungsten wires, etc. Each insulated electrical conductor is coupled to an electrical terminal in the electrical connector 252.

In certain embodiments, the tip 220 is removable and includes a fluidic connector to couple the aspiration tube 230 to the flexible aspiration tube 270, and electrical connectors to couple the aspiration tube 230 and the inner needle 240 to the insulated electrical conductors of the diathermy electrical cable 260.

In some embodiments, the cable 250 includes a flexible irrigating fluid tube, and the tip 220 includes an irrigating fluid tube to deliver irrigating fluid to the eye 10. The flexible irrigating fluid tube is coupled to the irrigating fluid tube, and to a fluidic connector at the proximal end of the cable 250. The fluidic connector may be coupled to the irrigation port 134. Alternatively, the aspiration tube 230 may be coupled to a valve that selects between the flexible irrigating fluid tube and the flexible aspiration tube 270. The footswitch 172 may also be used to control the irrigation function of the aspirating diathermy probe 200.

FIGS. 2B, 2C, 2D, and 2E depict views of the tip 220 of the aspirating diathermy probe 200, in accordance with certain embodiments of the present disclosure.

More particularly, FIG. 2B depicts a left side view of the distal end of the tip 220, FIG. 2C depicts a left side cross-sectional view of the distal end of the tip 220, FIG. 2D depicts a front view of the distal end of the tip 220, and FIG. 2E depicts a perspective view of the distal end of the tip 220.

The aspiration tube 230 has a distal end 232 that defines an opening 233 through which the aspirated material is received, an outer surface 234, and an inner surface 236 that defines a passageway 238 through which the aspirated material is removed. The proximal end of the aspiration tube 230 also defines an opening that is fluidically coupled to the flexible aspiration tube 270. The proximal end of the aspiration tube 230 is also electrically coupled to one of the insulated electrical conductors of the diathermy electrical cable 260. Generally, a distal portion of the outer surface 234, the edge of the aspiration tube 230, and a distal portion of the inner surface 236 provide one electrode surface for bipolar diathermy.

The inner needle 240 includes a conductor 242 and an insulating layer 248. The conductor 242 is made from an electrically conductive material, such as tungsten, copper, silver, stainless steel, etc., and the insulating layer 248 is made from an electrically insulating material, such as Polyvinyl Chloride (PVC), nylon, etc. The proximal end of the conductor 242 is electrically coupled to the other insulated electrical conductor of the diathermy electrical cable 260.

In certain embodiments, the conductor 242 has a conical tip 244 at the distal end, and a central core 246 that extends from the conical tip 244 to the proximal end of the conductor 242. The insulating layer 248 covers the central core 246, and the conical tip 244 provides the other electrode surface for bipolar diathermy.

The inner needle 240 is generally disposed within the passageway 238, with the distal end 241 of the inner needle 240 extending out of the passageway 238 past the distal end 232 of the aspiration tube 230. In certain embodiments, the insulating layer 248 may abut a portion of the inner surface 236 in a lower portion of the passageway 238 (as depicted). In other embodiments, the insulating layer 248 may abut a portion of the inner surface 236 in a left portion, a right portion, or an upper portion of the passageway 238. In some embodiments, the inner needle 240 may be centrally disposed within the passageway 238 such that the insulating layer 248 does not touch the inner surface 236.

In some embodiments, the inner needle 240 may translate along a longitudinal axis 249 from an extended position (as depicted) to a retracted position in which the conical tip 244 is disposed within the passageway 238. The retracted position prevents the inner needle 240 from contacting the tissue within the eye 10 and advantageously allows the opening 233 of the aspiration tube 230 to be positioned more closely to any particular location in the eye 10 by the surgeon. The inner needle 240 may be attached to an actuator (such as a slide, a toggle, etc.) on the handpiece to allow the surgeon to extend and retract the inner needle 240.

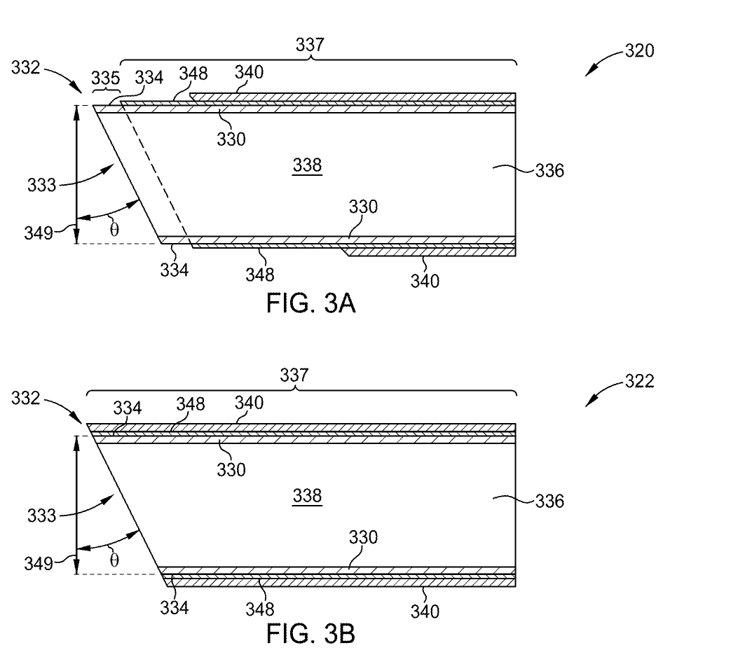

FIG. 3A depicts a left side cross-sectional view of the distal end of a tip 320 of a second example aspirating diathermy probe, in accordance with certain embodiments of the present disclosure. Generally, the second example aspirating diathermy probe includes the same components as the aspirating diathermy probe 200, with the tip 220 replaced by the tip 320.

In certain embodiments, the tip 320 includes, among other things, an aspiration tube 330, an outer tube 340, and an electrical insulating layer 348 disposed between the aspiration tube 330 and the outer tube 340. The distal end 332 of the aspiration tube 330 forms an angle θ with respect to the elevation axis 349 and defines an opening 333.

The aspiration tube 330 is made from an electrically conductive material. The aspiration tube 330 has an outer surface 334 and an inner surface 336 that defines an inner passageway 338 through which the aspirated material is removed. The proximal end of the aspiration tube 330 also defines an opening that is fluidically coupled to the flexible aspiration tube 270. The proximal end of the aspiration tube 330 is also electrically coupled to one of the insulated electrical conductors of the diathermy electrical cable 260. The aspiration tube 330 functions as one of the electrodes for bipolar diathermy.

The electrical insulating layer 348 covers most of the outer surface 334 (such as the insulated section 337) except for an exposed section 335 and the edge of the aspiration tube 330. The electrical insulating layer 348 is made from an electrically-insulating material, such as Polyvinyl Chloride (PVC), nylon, etc. Generally, the exposed section 335 of the outer surface 334, the edge of the aspiration tube 330, and a distal portion of the inner surface 336 provide one electrode surface for bipolar diathermy. The electrical insulating layer 348 may be applied to the aspiration tube 330 using various methods, such as by jet printing, etc. Generally, the thickness of the electrical insulating layer 348 may be about 4 to 6 microns, such as 5 microns. Other thicknesses may also be used, such as 6 to 10 microns, etc.

The outer tube 340 is electrically coupled to the other insulated electrical conductor of the diathermy electrical cable 260. The outer tube 340 is made from an electrically conductive material, and functions as the other electrode for bipolar diathermy. The outer tube 340 does not extend to the end of the aspiration tube 330.

FIG. 3B depicts a left side cross-sectional view of the distal end of a tip 322 of a variation of the second example aspirating diathermy probe, in accordance with certain embodiments of the present disclosure.

Generally, the tip 322 includes the same components as the tip 320. However, the insulating layer 348 covers all of the outer surface 334, which extends the insulated section 337 to the end of the aspirating tube 330 and removes the exposed section 335. The edge of the aspiration tube 330 and a distal portion of the inner surface 336 provide one electrode surface for bipolar diathermy. The outer tube 340 also extends to the end of the aspiration tube 330.

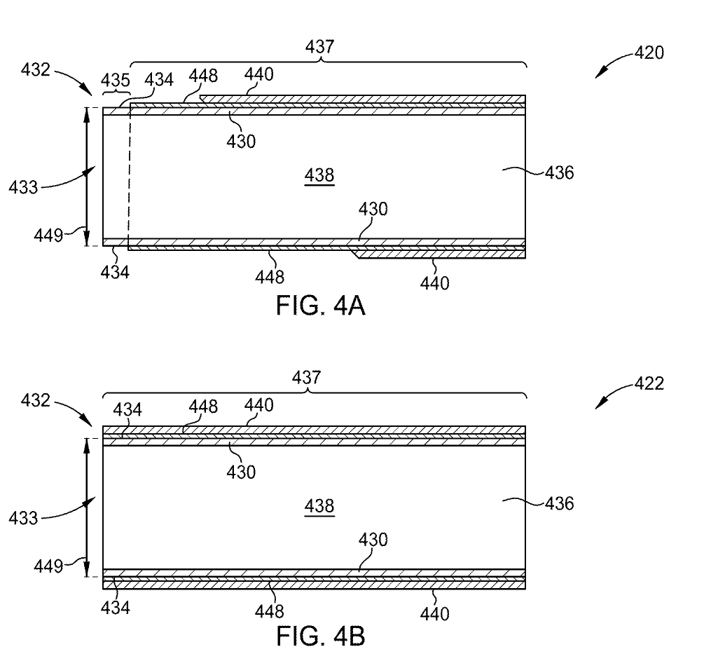

FIG. 4A depicts a left side cross-sectional view of the distal end of a tip 420 of a third example aspirating diathermy probe, in accordance with certain embodiments of the present disclosure.

Generally, the third example aspirating diathermy probe includes the same components as the aspirating diathermy probe 200, with the tip 220 replaced by the tip 420. Additionally, the tip 420 is the same as the tip 320 except for the angle of the distal end 432 of the aspiration tube 430.

In certain embodiments, the tip 420 includes, among other things, an aspiration tube 430, an outer tube 440, and an electrical insulating layer 448 disposed between the aspiration tube 430 and the outer tube 440. The distal end 432 of the aspiration tube 430 is parallel to the elevation axis 449 and defines an opening 433.

The aspiration tube 430 is made from an electrically conductive material, and has an outer surface 434, and an inner surface 436 that defines an inner passageway 438. The aspiration tube 430 functions as one of the electrodes for bipolar diathermy.

The electrical insulating layer 448 covers most of the outer surface 434 (such as the insulated section 437) except for an exposed section 435 of the aspiration tube 430. Generally, the exposed section 435 of the outer surface 434, the edge of the aspiration tube 430, and a distal portion of the inner surface 436 provide one electrode surface for bipolar diathermy.

The outer tube 440 is made from an electrically conductive material and functions as the other electrode for bipolar diathermy. The outer tube 440 does not extend to the end of the aspiration tube 430.

FIG. 4B depicts a left side cross-sectional view of the distal end of a tip 422 of a variation of the third example aspirating diathermy probe, in accordance with certain embodiments of the present disclosure.

Generally, the tip 422 is the same as the tip 322 except for the angle of the distal end 432 of the aspiration tube 430. The insulating layer 448 covers all of the outer surface 434, which extends the insulated section 437 to the end of the aspirating tube 430 and removes the exposed section 435. The edge of the aspiration tube 430 and a distal portion of the inner surface 436 provide one electrode surface for bipolar diathermy. The outer tube 440 also extends to the edge of the aspiration tube 430.

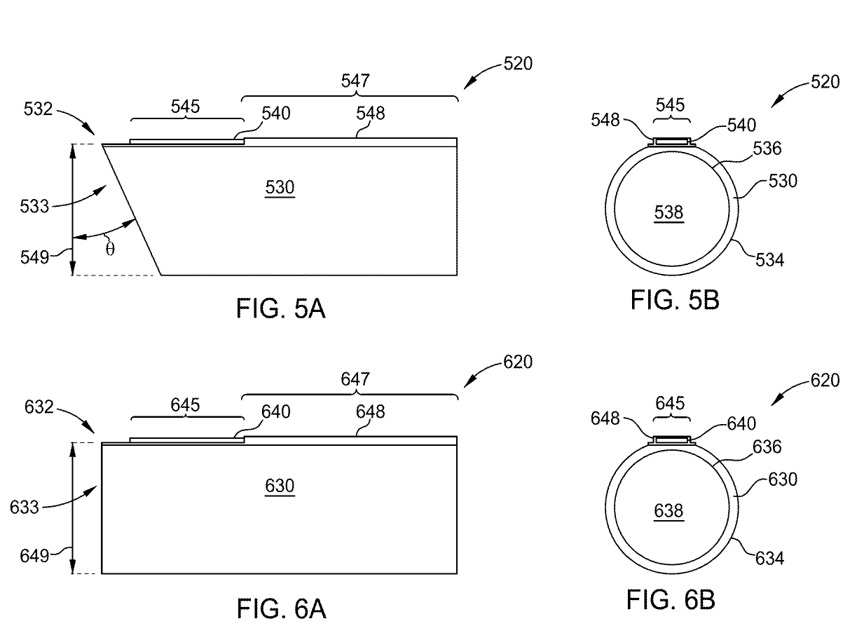

FIGS. 5A and 5B depict a left side view and a front view (respectively) of the distal end of a fourth example aspirating diathermy probe, in accordance with certain embodiments of the present disclosure. Generally, the fourth example aspirating diathermy probe includes the same components as the aspirating diathermy probe 200, with the tip 220 replaced by the tip 520.

In certain embodiments, the tip 520 includes, among other things, an aspiration tube 530, a conductor strip 540 disposed on the aspiration tube 530, and an electrical insulating layer 548 disposed between the aspiration tube 530 and the conductor strip 540. The distal end 532 of the aspiration tube 530 forms an angle θ with respect to the elevation axis 549 and defines an opening 533.

The aspiration tube 530 is made from electrically conductive material, and has an outer surface 534, and an inner surface 536 that defines an inner passageway 538. At least a portion of the inner surface 536 functions as one electrode for bipolar diathermy.

The electrical insulating layer 548 covers an upper portion of the outer surface 534. The electrical insulating layer 548 also covers most of the conductor strip 540 (such as the insulated section 547) except for an exposed section 545 of the conductor strip 540 which does not extend to the edge of the distal end 532.

The conductor strip 540 is made from an electrically conductive material and is electrically coupled to the other insulated electrical conductor of the diathermy electrical cable 260. The exposed section 545 of the conductor strip 540 forms the other electrode for bipolar diathermy.

The electrical insulating layer 548 and the conductor strip 540 may be applied to the aspiration tube 530 using various methods.

In certain embodiments, a flat surface or plane is machined along the axial length of the outer surface 534, and a first portion of the electrical insulating layer 548 is applied to the flat surface, such as by jet printing, etc. The conductor strip 540 is then applied to the electrical insulating layer 548, such as by jet printing, etc. A second portion of the electrical insulating layer 548 is then applied to the conductor strip 540, such as by jet printing, etc., leaving the exposed section 545 of the conductor strip 540 uninsulated. The exposed section 545 of the conductor strip 540 may form a rectangular shape (as depicted). Other shapes may also be used, such as a triangular shape, etc. Generally, the thickness of the first and second portions of the electrical insulating layer 548 and the conductor strip 540 may be about 4 to 6 microns, such as 5 microns. Other thicknesses may also be used, such as 6 to 10 microns, etc.

FIGS. 6A and 6B depict a left side view and a front view (respectively) of the distal end of a fifth example aspirating diathermy probe, in accordance with certain embodiments of the present disclosure. Generally, the fifth example aspirating diathermy probe includes the same components as the aspirating diathermy probe 200, with the tip 220 replaced by the tip 620. Additionally, the tip 620 is the same as the tip 520 except for the angle of the distal end 632 of the aspiration tube 630.

In certain embodiments, the tip 620 includes, among other things, an aspiration tube 630, a conductor strip 640 disposed on the aspiration tube 630, and an electrical insulating layer 648 disposed between the aspiration tube 630 and the conductor strip 640. The distal end 632 of the aspiration tube 630 is parallel to the elevation axis 649 and defines an opening 633.

The aspiration tube 630 is made from electrically conductive material and has an outer surface 634 and an inner surface 636 that defines an inner passageway 638. At least a portion of the inner surface 636 functions as one electrode for bipolar diathermy.

The electrical insulating layer 648 covers an upper portion of the outer surface 634. The electrical insulating layer 648 also covers most of the conductor strip 640 (such as the insulated section 647) except for an exposed section 645 of the conductor strip 640 which does not extend to the edge of the distal end 632.

The conductor strip 640 is made from an electrically conductive material, and the exposed section 645 of the conductor strip 640 forms the other electrode for bipolar diathermy.

The electrical insulating layer 648 and the conductor strip 640 may be applied to the aspiration tube 630 using various methods, as described above with respect to the electrical insulating layer 548 and the conductor strip 540.

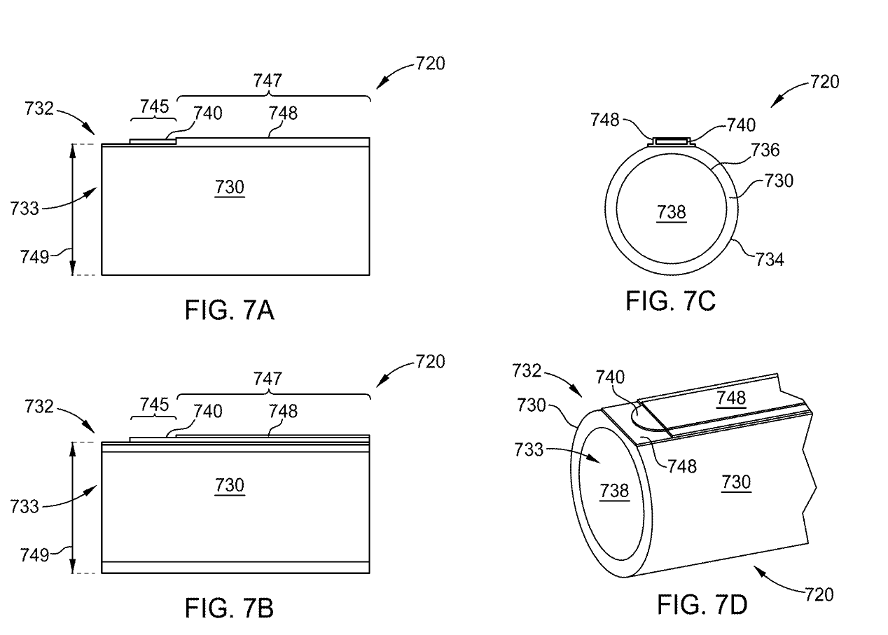

FIGS. 7A, 7B, 7C and 7D, depict views of the tip 720 of the sixth example aspirating diathermy probe, in accordance with certain embodiments of the present disclosure.

More particularly, FIG. 7A depicts a left side view of the distal end of the tip 720, FIG. 7B depicts a left side cross-sectional view of the distal end of the tip 720, FIG. 7C depicts a front view of the distal end of the tip 720, and FIG. 7D depicts a perspective view of the distal end of the tip 720. Generally, the sixth example aspirating diathermy probe includes the same components as the aspirating diathermy probe 200, with the tip 220 replaced by the tip 720. Additionally, the tip 720 is the same as the tip 620 except for the length and semicircular shape of the exposed section 745.

In certain embodiments, the tip 720 includes, among other things, an aspiration tube 730, a conductor strip 740 disposed on the aspiration tube 730, and an electrical insulating layer 748 disposed between the aspiration tube 730 and the conductor strip 740. The distal end 732 of the aspiration tube 730 is parallel to the elevation axis 749 and defines an opening 733.

The aspiration tube 730 is made from an electrically conductive material, has an outer surface 734, and an inner surface 736 that defines an inner passageway 738. At least a portion of the inner surface 736 functions as one electrode for bipolar diathermy.

The electrical insulating layer 748 covers an upper portion of the outer surface 734. The electrical insulating layer 748 also covers most of the conductor strip 740 (such as the insulated section 747) except for an exposed section 745 of the conductor strip 740 which does not extend to the edge of the distal end 732.

The conductor strip 740 is made from an electrically conductive material, and the exposed section 745 of the conductor strip 740 forms the other electrode for bipolar diathermy.

The electrical insulating layer 748 and the conductor strip 740 may be applied to the aspiration tube 730 using various methods, as described above with respect to the electrical insulating layer 548 and the conductor strip 540.

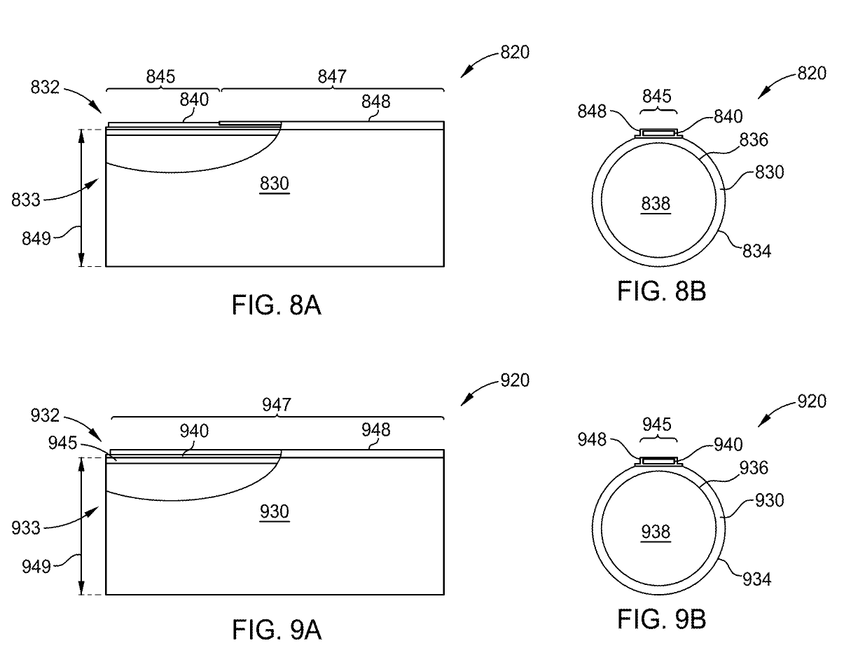

FIGS. 8A and 8B depict a left side view (partial cutaway) and a front view (respectively) of the distal end of a seventh example aspirating diathermy probe, in accordance with certain embodiments of the present disclosure. Generally, the seventh example aspirating diathermy probe includes the same components as the aspirating diathermy probe 200, with the tip 220 replaced by the tip 820. Additionally, the tip 820 is the same as the tip 620 except that the exposed section 845 extends to the edge of the distal end 832.

In certain embodiments, the tip 820 includes, among other things, an aspiration tube 830, a conductor strip 840 disposed on the aspiration tube 830, and an electrical insulating layer 848 disposed between the aspiration tube 830 and the conductor strip 840. The distal end 832 of the aspiration tube 830 is parallel to the elevation axis 849 and defines an opening 833.

The aspiration tube 830 is made from electrically conductive material, has an outer surface 834, and an inner surface 836 that defines an inner passageway 838. At least a portion of the inner surface 836 functions as one electrode for bipolar diathermy.

The electrical insulating layer 848 covers an upper portion of the outer surface 834. The electrical insulating layer 848 also covers most of the conductor strip 840 (such as the insulated section 847) except for an exposed section 845 of the conductor strip 840 which extends to the edge of the distal end 832.

The conductor strip 840 is made from an electrically conductive material, and the exposed section 845 of the conductor strip 840 forms the other electrode for bipolar diathermy.

The electrical insulating layer 848 and the conductor strip 840 may be applied to the aspiration tube 830 using various methods, as described above with respect to the electrical insulating layer 548 and the conductor strip 540.

FIGS. 9A and 9B depict a left side view (partial cutaway) and a front view (respectively) of the distal end of an eighth example aspirating diathermy probe, in accordance with certain embodiments of the present disclosure. Generally, the eighth example aspirating diathermy probe includes the same components as the aspirating diathermy probe 200, with the tip 220 replaced by the tip 920. Additionally, the tip 920 is the same as the tip 820 except that the insulated section 947 extends to the edge of the distal end 932, and a small rectangular strip of the conductor strip 940 is uninsulated.

In certain embodiments, the tip 920 includes, among other things, an aspiration tube 930, a conductor strip 940 disposed on the aspiration tube 930, and an electrical insulating layer 948 disposed between the aspiration tube 930 and the conductor strip 940. The distal end 932 of the aspiration tube 930 is parallel to the elevation axis 949 and defines an opening 933.

The aspiration tube 930 is made from an electrically conductive material, and has an outer surface 934, and an inner surface 936 that defines an inner passageway 938. At least a portion of the inner surface 936 functions as one electrode for bipolar diathermy.

The electrical insulating layer 948 covers an upper portion of the outer surface 934. The electrical insulating layer 948 also covers the conductor strip 940 (such as the insulated section 947, which extends to the edge of the distal end 932). Only the end of the conductor strip 940 is exposed (such as the exposed section 945).

The conductor strip 940 is made from an electrically conductive material, and the exposed section 945 of the conductor strip 940 forms the other electrode for bipolar diathermy.

The electrical insulating layer 948 and the conductor strip 940 may be applied to the aspiration tube 930 using various methods, as described above with respect to the electrical insulating layer 548 and the conductor strip 540.

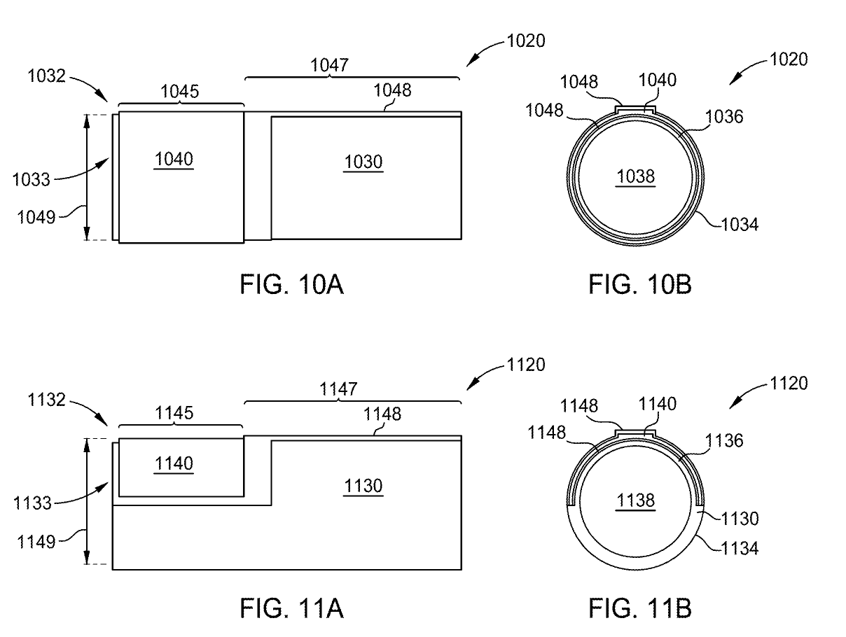

FIGS. 10A and 10B depict a left side view and a front view (respectively) of the distal end of a ninth example aspirating diathermy probe, in accordance with certain embodiments of the present disclosure. Generally, the ninth example aspirating diathermy probe includes the same components as the aspirating diathermy probe 200, with the tip 220 replaced by the tip 1020. Additionally, the tip 1020 is the same as the tip 620 with the addition of the ring-shaped surface of the conductor strip 1040 and the corresponding electrical insulating layer 1048 covering the distal end 1032 of the aspiration tube 1030.

In certain embodiments, the tip 1020 includes, among other things, an aspiration tube 1030, a conductor strip 1040 disposed on the aspiration tube 1030, and an electrical insulating layer 1048 disposed between the aspiration tube 1030 and the conductor strip 1040. The distal end 1032 of the aspiration tube 1030 is parallel to the elevation axis 1049 and defines an opening 1033.

The aspiration tube 1030 is made from electrically conductive material, and has an outer surface 1034, and an inner surface 1036 that defines an inner passageway 1038. At least a portion of the inner surface 1036 functions as one electrode for bipolar diathermy.

The electrical insulating layer 1048 covers an upper portion of the outer surface 1034 and the distal end 1032 of the aspiration tube 1030. The electrical insulating layer 1048 also covers the conductor strip 1040 (such as the insulated section 1047, which extends to the edge of the distal end 1032). A ring-shaped portion of the conductor strip 1040 is exposed (such as the exposed section 1045).

The conductor strip 1040 is made from an electrically conductive material, and the exposed section 1045 of the conductor strip 1040 forms the other electrode for bipolar diathermy.

The electrical insulating layer 1048 and the conductor strip 1040 may be applied to the aspiration tube 1030 using various methods, as described above with respect to the electrical insulating layer 548 and the conductor strip 540.

FIGS. 11A and 11B depict a left side view and a front view (respectively) of the distal end of a tenth example aspirating diathermy probe, in accordance with certain embodiments of the present disclosure. Generally, the tenth example aspirating diathermy probe includes the same components as the aspirating diathermy probe 200, with the tip 220 replaced by the tip 1120. Additionally, the tip 1120 is the same as the tip 1020 with a partial, ring-shaped portion of conductor strip 1140.

In certain embodiments, the tip 1120 includes, among other things, an aspiration tube 1130, a conductor strip 1140 disposed on the aspiration tube 1130, and an electrical insulating layer 1148 disposed between the aspiration tube 1130 and the conductor strip 1140. The distal end 1132 of the aspiration tube 1130 is parallel to the elevation axis 1149 and defines an opening 1133.

The aspiration tube 1130 is made from electrically conductive material, and has an outer surface 1134, and an inner surface 1136 that defines an inner passageway 1138. At least a portion of the inner surface 1136 functions as one electrode for bipolar diathermy.

The electrical insulating layer 1148 covers an upper portion of the outer surface 1134 and the distal end 1132 of the aspiration tube 1130. The electrical insulating layer 1148 also covers the conductor strip 1140 (such as the insulated section 1147). A partial ring-shaped portion of the conductor strip 1140 is exposed (such as the exposed section 1145).

The conductor strip 1140 is made from an electrically conductive material, and the exposed section 1145 of the conductor strip 1140 forms the other electrode for bipolar diathermy.

The electrical insulating layer 1148 and the conductor strip 1140 may be applied to the aspiration tube 1130 using various methods, as described above with respect to the electrical insulating layer 548 and the conductor strip 540.

### Example Embodiments

In certain embodiments, a surgical device comprises a flexible aspiration tube, a diathermy electrical cable, a handpiece, and a tip attached to the handpiece. The handpiece is configured to support a distal portion of the flexible aspiration tube and a distal portion of the diathermy electrical cable. The tip comprises an aspiration tube coupled to the distal portion of the flexible aspiration tube and the distal portion of the diathermy electrical cable, and an inner needle coupled to the distal portion of the diathermy electrical cable. The aspiration tube is a first diathermy electrode, and the inner needle is a second diathermy electrode.

In certain other embodiments, a surgical device comprises a flexible aspiration tube, a diathermy electrical cable, a handpiece, and a tip attached to the handpiece. The handpiece is configured to support a distal portion of the flexible aspiration tube and a distal portion of the diathermy electrical cable. The tip comprises an aspiration tube coupled to the distal portion of the flexible aspiration tube and the distal portion of the diathermy electrical cable, an outer tube coupled to the distal portion of the diathermy electrical cable, and an electrical insulating layer disposed between the aspiration tube and the outer tube. The aspiration tube is a first diathermy electrode, and the outer tube is a second diathermy electrode.

In certain other embodiments, a surgical device comprises a flexible aspiration tube, a diathermy electrical cable, a handpiece, and a tip attached to the handpiece. The handpiece is configured to support a distal portion of the flexible aspiration tube and a distal portion of the diathermy electrical cable. The tip comprises an aspiration tube coupled to the distal portion of the flexible aspiration tube and the distal portion of the diathermy electrical cable, a conductor strip disposed on the aspiration tube and coupled to the distal portion of the diathermy electrical cable, and an electrical insulating layer disposed between the aspiration tube and the conductor strip. The aspiration tube is a first diathermy electrode, and the conductor strip is a second diathermy electrode.

The many features and advantages of the disclosure are apparent from the detailed specification, and, thus, it is intended by the appended claims to cover all such features and advantages of the disclosure which fall within the scope of the disclosure. Further, since numerous modifications and variations will readily occur to those skilled in the art, it is not desired to limit the disclosure to the exact construction and operation illustrated and described, and, accordingly, all suitable modifications and equivalents may be resorted to that fall within the scope of the disclosure.

What is claimed is:

1. A surgical device, comprising:
a flexible aspiration tube;
a diathermy electrical cable;
a handpiece configured to support a distal portion of the flexible aspiration tube and a distal portion of the diathermy electrical cable; and
a tip attached to the handpiece, the tip comprising:
an aspiration tube coupled to the distal portion of the flexible aspiration tube and the distal portion of the diathermy electrical cable, and
an inner needle coupled to the distal portion of the diathermy electrical cable,
wherein:
the aspiration tube is a first diathermy electrode, and
the inner needle is a second diathermy electrode.

2. The surgical device of claim 1, wherein:
the aspiration tube comprises:
a distal end defining a distal opening through which aspirated material is received,
an outer surface,
an inner surface defining a passageway through which the aspirated material is removed, and
a proximal end defining a proximal opening, the proximal end coupled to the distal portion of the flexible aspiration tube and the distal portion of the diathermy electrical cable; and
at least a portion of the outer surface and a portion of the inner surface form the first diathermy electrode.

3. The surgical device of claim 2, wherein:
the inner needle comprises:
a conductor including a conical tip and a central core, and
an insulating layer covering the central core; and
the conical tip is the second diathermy electrode.

4. The surgical device of claim 3, wherein:
a portion of the inner needle is disposed within the passageway of the aspiration tube; and
the conical tip of the conductor is disposed outside the passageway of the aspiration tube.

5. The surgical device of claim 4, wherein:
the inner needle is movable between an extended position and a retracted position;
in the extended position, the conical tip of the conductor is disposed outside the passageway of the aspiration tube; and
in the retracted position, the conical tip of the conductor is disposed inside the passageway of the aspiration tube.

6. The surgical device of claim 3, wherein the diathermy electrical cable comprises:
a first insulated conductor coupled to the aspiration tube; and
a second insulated conductor coupled to the conductor of the inner needle.

7. The surgical device of claim 6, wherein:
the diathermy electrical cable further comprises an electrical connector coupled to the first and second insulated conductors; and
the electrical connector is disposed at a proximal end of the diathermy electrical cable.

8. The surgical device of claim 1, further comprising:
a cable comprising the flexible aspiration tube and the diathermy electrical cable.

9. The surgical device of claim 1, wherein:
the handpiece comprises a passageway through which the distal portion of the flexible aspiration tube and the distal portion of the diathermy electrical cable extend.

10. The surgical device of claim 1, wherein the tip is removable.

11. A surgical device, comprising:
a diathermy electrical cable;
a flexible aspiration tube;
a handpiece configured to support a distal portion of the diathermy electrical cable and a distal portion of the flexible aspiration tube; and
a tip attached to the handpiece, the tip comprising:
an aspiration tube coupled to the distal portion of the flexible aspiration tube and the distal portion of the diathermy electrical cable, and
an outer tube coupled to the distal portion of the diathermy electrical cable, and
an electrical insulating layer disposed between the aspiration tube and the outer tube,
wherein:
the aspiration tube is a first diathermy electrode, and
the outer tube is a second diathermy electrode.

12. The surgical device of claim 11, wherein:
the aspiration tube comprises:
a distal end comprising an edge defining a distal opening through which aspirated material is received,
an outer surface having an insulated section that is covered by the electrical insulating layer and an exposed section that is not covered by the electrical insulating layer,
an inner surface defining a passageway through which the aspirated material is removed, and
a proximal end defining a proximal opening, the proximal end coupled to the distal portion of the flexible aspiration tube and the distal portion of the diathermy electrical cable; and
the exposed section of the outer surface, the edge of the aspiration tube, and at least a portion of the inner surface form the first diathermy electrode.

13. The surgical device of claim 12, wherein the exposed section of the outer surface extends from an end of the electrical insulating layer to the edge of the aspiration tube.

14. The surgical device of claim 12, wherein:
the exposed section of the outer surface is covered by the insulated section; and
the edge of the aspiration tube and the portion of the inner surface form the first diathermy electrode.

15. The surgical device of claim 14, wherein the outer tube extends to the edge of the aspiration tube.

16. A surgical device, comprising:
a diathermy electrical cable;
a flexible aspiration tube;
a handpiece configured to support a distal portion of the diathermy electrical cable and a distal portion of the flexible aspiration tube; and
a tip attached to the handpiece, the tip comprising:
an aspiration tube coupled to the distal portion of the flexible aspiration tube and the distal portion of the diathermy electrical cable, and
a conductor strip disposed on the aspiration tube, the conductor strip coupled to the distal portion of the diathermy electrical cable, and
an electrical insulating layer disposed between the aspiration tube and the conductor strip,
wherein:
the aspiration tube is a first diathermy electrode, and
the conductor strip is a second diathermy electrode.

17. The surgical device of claim 16, wherein:
the electrical insulating layer partially covers the conductor strip to form an insulated section that is covered by the electrical insulating layer and an exposed section that is not covered by the electrical insulating layer;
the aspiration tube comprises:
a distal end comprising an edge defining a distal opening through which aspirated material is received,
an outer surface partially covered by the electrical insulating layer,
an inner surface defining a passageway through which the aspirated material is removed, and
a proximal end defining a proximal opening, the proximal end coupled to the distal portion of the flexible aspiration tube and the distal portion of the diathermy electrical cable;
at least a portion of the inner surface forms the first diathermy electrode; and
the exposed section of the conductor strip forms the second diathermy electrode.

18. The surgical device of claim 17, wherein the exposed section of the conductor strip does not extend to the edge of the aspiration tube.

19. The surgical device of claim 17, wherein the exposed section of the conductor strip extends to the edge of the aspiration tube.

20. The surgical device of claim 17, wherein the exposed section of the conductor strip forms at least a partial ring around the distal end of the aspiration tube.
